(19) United States
(12) Patent Application Publication
(10) Pub. No.: US 2010/0210951 A1
(43) Pub. Date: Aug. 19, 2010

(54) Optical System for Imaging of Tissue Lesions

(76) Inventors: Mohammed Saidur Rahman, Dallas, TX (US); Rebecca Rae Richards-Kortum, Houston, TX (US); Ann M. Gillenwater, Pearland, TX (US)

Correspondence Address: Baker Botts L.L.P, 910 Louisiana Street, One Shell Plaza, HOUSTON, TX, 77002, US

(21) Appl. No.: 12/638,459

(22) Filed: Dec. 15, 2009

Related U.S. Application Data
(63) Continuation of application No. PCT/US2007/071296, filed on Jun. 15, 2007.

(51) Int. Cl. A61B 6/00

(52) U.S. Cl. 600/476

(57) ABSTRACT

The present disclosure, according to one embodiment, relates to an optical device for direct visualization, imaging, and spectroscopic measurements of tissue abnormalities at various anatomical sites. Such sites include, but are not limited to, the oral cavity, the cervix, and the skin. In one embodiment, the device comprises: a frame; at least one light-emitting diode light source mounted on the frame for illuminating a tissue, wherein the at least one light-emitting diode light source is chosen from a fluorescent light source, a polarized white light source, and an unpolarized white light source; at least one loupe mounted on the frame for visually observing the tissue; at least one filter disposed between the tissue and the loupe for filtering light reflected from the tissue; and an energy source operably connected to the at least one light-emitting diode light source.

### CROSS-REFERENCE TO RELATED APPLICATIONS

This application is a continuation of International Application No. PCT/US2007/071296 filed Jun. 15, 2007, which is incorporated herein by reference.

### STATEMENT OF GOVERNMENT INTEREST

This disclosure was developed at least in part using funding from the National Institute of Dental and Craniofacial Research, Grant No. 5R21DE016485. The U.S. government may have certain rights in the invention.

### BACKGROUND

Imaging of tissue lesions has become an increasingly important medical technology for the diagnosis and treatment of a variety of pathological conditions. For example, Oral cancer is a major health problem in some parts of the world, especially in developing countries. According to the World Health Organization (WHO), the worldwide annual incidence for oral cancer exceeds 267,000 new cases with an estimated 128,000 deaths, nearly two-thirds of which is observed in developing countries [1]. The WHO further predicts a continuing worldwide increase in incidence for the next several decades. In the US, it is estimated that approximately 34,360 people will be diagnosed with oral cancer and another 7,550 people will succumb to the disease in 2007 [2]. These numbers will also likely rise due to increased immigration to the US from high-risk countries.

The 5-year survival rate for oral cancer is one of the lowest of all major cancers. In the U.S., only 54% of all patients with oral cancer live for five years or more after the initial diagnosis [2]. The survival rate drops below 30% for developing countries mainly due to the lack of awareness, inadequate screening programs, and inability to detect disease in early stages [3]. Although it has been shown that early detection can improve the 5-year survival rate to 80%, there has been very little improvement over the last three decades on the survival outcome of oral cancer. For the vast majority of cases, the disease is diagnosed late at an advanced stage, requiring more aggressive treatment and still resulting in poor survival and increased morbidity. Thus, detecting oral cancer at an early stage remains key to improving survival outcome of the disease and quality of life for patients.

The standard method for screening and detection of oral neoplasia is visual inspection of oral cavity under white light. Once identified, suspicious lesions must be biopsied and undergo histological evaluation to determine the presence and extent of the disease. Clinical manifestations such as white patches (leukoplakia) and red patches (erythroplakia), are assessed during the visual examination to mark suspected lesions [4]. Unfortunately, even for experienced physicians these clinical signatures are difficult to differentiate from nonspecific inflammation and irritation which also appears as white or red patches. Furthermore, many lesions appear clinically occult, which can also result in a failure to biopsy. Although biopsy can be used as an alternative screening method, it is not a practical solution as it can both result in patient discomfort and accrue the cost especially when screening large populations in developing countries. Altogether, a non-invasive low-cost screening tool is needed that is both sensitive and specific and can be easily translated to the poor resource settings in developing countries.

A variety of approaches have been utilized to perform improved diagnoses of oral cancer and other skin lesions. Digital processing of reflectance images, while improving the registration, recording, and documentation of skin lesions, has not provided any improvement in diagnostic accuracy over while light visualization. Ultraviolet and infrared photography have also been utilized in these diagnostic procedures, but delays in film image processing make these techniques impractical. Mercury discharge lamps offer an improvement over ultraviolet light sources for these applications because the fluorescent light reflected from the tissue can be seen with the eye, but they must be used in a darkened room. Additionally, all of these devices comprise technology or energy requirements that render them impractical for use in underdeveloped settings. These techniques are described in U.S. Pat. No. 6,021,344, the relevant disclosures of which are hereby incorporated herein by reference.

Fluorescence imaging has been shown to be an effective alternative method for screening and diagnosis of pre-cancers in several organ sites including oral cavity, cervix, lung and skin [5-10]. Several groups including Betz et al, Onizawa et al, Paczona et al and Sivstun et al have shown that examining the oral cavity under a fluorescence excitation light source can overcome some of the detection limitations associated with standard white light examination. Betz et al and Paczona et al used a xenon arc lamp as a light source to excite tissue at wavelengths between 375-440 nm and detect the autofluorescence signals above 515 nm using a color CCD [7, 8]. Onizawa et al used an UV flash lamp with an illumination peak at 360 nm to induce porphyrin-like fluorescence at 630 nm and recorded signals on photographic film [9]. Later, Sivstun et al conducted a study to find the optimal excitation and emission wavelengths for direct visualization of oral cavity for differentiating normal tissue from neoplasia [10]. Lane et al recently proposed a simple hand-held device for direct visualization of tissue autofluorescence above 480 nm using a metal halide mercury lamp with excitation wavelengths between 360-460 nm [11]. The device is currently approved for medical use by the Food and Drug Administration (FDA) in the U.S. All of these studies highlight the fact that examining the autofluorescence signal of the oral cavity under fluorescence excitation wavelengths between 360-460 nm can be a powerful tool for screening oral cancer.

Although previous fluorescence imaging devices have shown high sensitivity and specificity for detecting abnormalities in the oral cavity, their use has been mainly limited to medical facilities in the developed countries. They are a less practical solution for mass screening of the high-risk populations in low-resource settings as the cost of these devices is relatively high, their portability is limited, and all of them require a stable high-voltage power supply. Furthermore, these devices cannot be used additionally for traditional white light examination which may prevent clinicians from obtaining clinical impressions they are accustomed to observing.

While the background of the present disclosure is intimately related with the imaging of the oral cavity, such a motivation is not intended to be limiting; rather, the system and method embodied in the present disclosure can be used to visualize a variety of externally accessible tissues.

### SUMMARY

The present disclosure, according to one embodiment, relates to a device for examining tissue comprising: a frame, at least one light-emitting diode light source mounted on the frame for illuminating a tissue, wherein the at least one light-emitting diode light source is chosen from a fluorescent light source, a polarized white light source, or an unpolarized white light source, at least one loupe mounted on the frame for visually observing the tissue, at least one filter disposed between the tissue and the loupe for filtering light reflected from the tissue, and an energy source operably connected to the at least one light-emitting diode light source. In some embodiments, the device may also comprise a camera mounted on the frame for capturing an image of the tissue. In some embodiments, the device may also comprise a display monitor operably connected to the camera for receiving a signal produced by the came. The camera may be any suitable camera for recording images of tissue under the above mentioned light conditions. The energy source may be any energy source suitable to power the optical device for a desired amount of time.

In another embodiment, the present disclosure relates to a method for visualizing tissue comprising: illuminating a tissue with at least one light-emitting diode light source chosen from a fluorescent light source, a polarized white light source, and an unpolarized white light source; and observing the tissue after illumination with the at least one light-emitting diode light source.

The features and advantages of the present disclosure will be readily apparent to those skilled in the art upon a reading of the description of the embodiments that follows.

### DRAWINGS

A more complete understanding of this disclosure may be acquired by referring to the following description taken in combination with the accompanying drawings.

FIG. 1 is an example construction of an optical device of the present disclosure.

FIG. 2A shows the beam pattern of fluorescence illumination of a device of the present disclosure.

FIG. 2B shows the beam uniformity of a device of the present disclosure.

FIG. 3A is floor of mouth images of a normal volunteer captured by an optical device of the present disclosure under fluorescence.

FIG. 3B is floor of mouth images of a normal volunteer captured by an optical device of the present disclosure under un-polarized white light reflectance.

FIG. 3C is floor of mouth images of a normal volunteer captured by an optical device of the present disclosure under polarized white light reflectance.

FIG. 4A is floor of mouth images of a patient captured by an optical device of the present disclosure under fluorescence.

FIG. 4B is floor of mouth images of a patient captured by an optical device of the present disclosure under un-polarized white light reflectance.

FIG. 4C is floor of mouth images of a patient captured by an optical device of the present disclosure under polarized white light reflectance.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to one embodiment, relates to a device for examining tissue comprising: a frame; at least one light-emitting diode light source mounted on the frame for illuminating a tissue, wherein the at least one light-emitting diode light source is selected from the group consisting of: a fluorescent light source, a polarized white light source, and an unpolarized white light source; at least one loupe mounted on the frame for visually observing the tissue; at least one filter disposed between the tissue and the loupe for filtering light reflected from the tissue; and an energy source operably connected to the at least one light-emitting diode light source. Optionally, the device may also comprise a camera mounted on the frame for capturing images of the tissue or a display monitor operably connected to the camera for receiving a signal produced by the camera.

The light source utilized in the device of the present disclosure may be a fluorescent or white light source. Appropriate white light sources include both polarized and unpolarized white light sources. Preferably, the light sources of the present disclosure are in the form of light-emitting diodes (LEDs) so as to minimize the size, weight, and energy consumption of the device. An example of an LED that may be used in the device of the present disclosure is the Luxeon Royal Blue-K2 LED. An example of a white light source that may be used in the device of the present disclosure is the Heine LoupeLight.

The device of the present disclosure may optionally comprise a camera. Suitable cameras for this device include any camera capable of capturing images in a field provided by the fluorescent or polarized or unpolarized white light sources described above. An example of such a camera is a charge-coupled device (CCD) camera such as the Prosilica EC1380C.

The energy source utilized in the device of the present disclosure can be any energy source capable of supplying energy to the device for an adequate period of time. In preferred embodiments, the energy source is a battery. In more preferred embodiments, the energy source is a lithium-ion battery.

To facilitate a better understanding of the present disclosure, the following examples of specific embodiments are given. In no way should the following examples be read to limit or define the entire scope of the invention.

### EXAMPLES

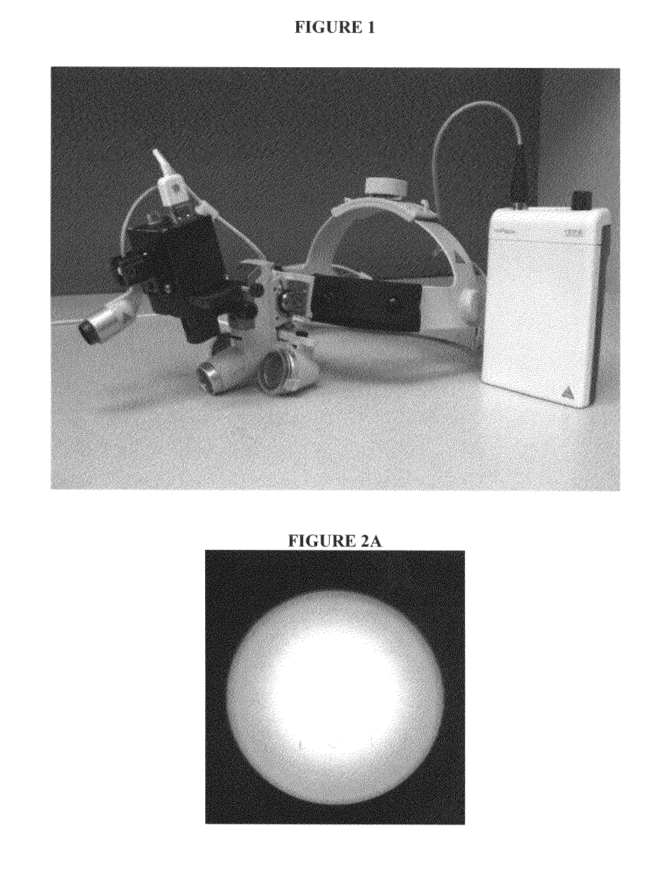

An example embodiment of the system of the present disclosure is shown in FIG. 1. In one embodiment, an optical device of the present disclosure weighs only three pounds and consists of a commercially available surgical head-light with loupes (Heine USA Ltd.), a light emitting diode, a remote-head CCD camera (Prosilica EC1380C), and a lithium-ion battery. The 2.5× magnification binocular loupes provided a working distance (WD) of 250 mm, field of view (FOV) of 55 mm, and depth of field (DOF) of 55 mm. The resolution of the loupe was tested with a U.S. Air Force resolution target and up to 4 line pairs per millimeter could be resolved. The head-light system was modified to provide excitation light for fluorescence imaging with a blue LED (Luxeon Royal Blue-K2). The 750 mW rated LED provides a peak irradiance of 15 mW/cm² at the center of the measurement site with peak intensity at 455 nm. An additional light source (Heine LoupeLight) was incorporated to the system for white light illumination. A rechargeable lithium-ion battery provided with the optical device was used for powering the light sources and can be used continuously for four hours. The camera was powered and controlled by a laptop computer through an IEEE 1394 Firewire port. Images of objects on the camera were made parfocal with binocular loupes using a focus adjustable c-mount lens.

In order to examine and record tissue signals in different modalities including fluorescence, white light (un-polarized) and polarized white light mode, appropriate optical components were introduced in the optical light path. For fluorescence illumination, a 447/60 nm excitation filter (Semrock FF02-447/60) was placed in front of the blue LED to prevent any bleed-through above 480 nm. The emitted signals could be observed through the binocular loupes which had single 480 nm long pass emission filters (Omega Optical 480ALP) attached in front of each loupe to block fluorescence excitation light. Similarly, for polarized light reflectance, a polarizer was placed in front of the white LED light; an additional polarizer oriented at 90° relative to the first can be placed in the detection light path to remove any specular reflection if desired. The filter and polarizer in the detection path of the camera were placed using a custom designed filter holder. This holder contains three positions to allow different optical components to be easily interchanged during patient examinations in different imaging modes.

Since uniformity of light illumination can influence the perceived contrast of measured objects, the beam pattern of the fluorescence light and white light were characterized prior to taking any tissue images. The illumination profile of the optical device was measured on a uniform diffusive surface with the integrated CCD camera. In order to avoid interference from ambient light, all images including patient measurements were acquired in a relatively dark room. Images were recorded in a programmed graphical user interface software in LabView. All imaging measurements on humans were taken with prior consent from the subjects and according to a protocol approved by the Institutional Review Board (IRB) at Rice University and MD Anderson Cancer Center.

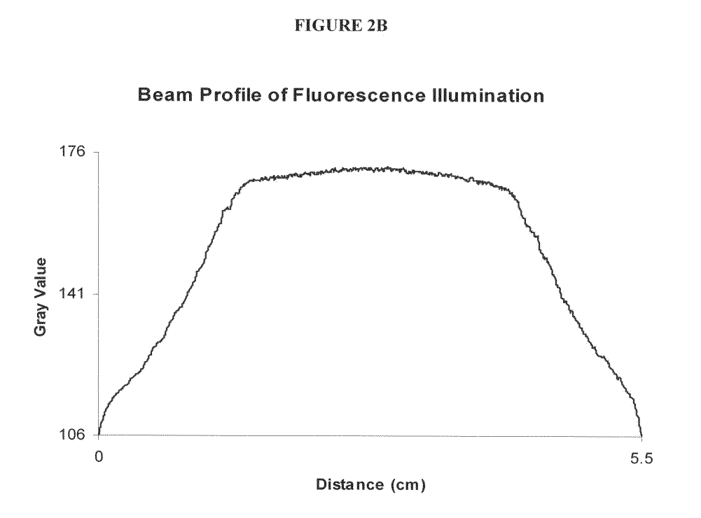

In FIG. 2a, beam pattern of the fluorescence illumination across the field of view is represented in grayscale value with white indicating a region of higher light intensity and black indicating no light. The cross-section of the beam pattern is shown in FIG. 2b to indicate the uniformity of illumination across the field. The white light illumination was also characterized in a similar fashion and revealed similar beam pattern.

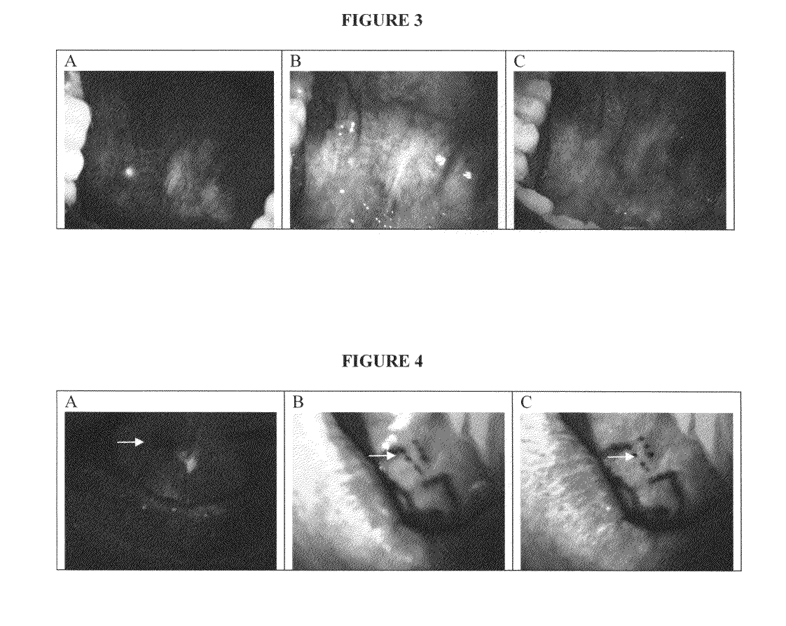

FIG. 3 shows images of the floor of mouth of a normal subject using different imaging modes of the portable screening system. Although the bright fluorescence signal from the teeth partially saturates the image, green autofluorescence signal from tissue is clearly visible in FIG. 3A. Similarly, in the standard white light image (un-polarized), as shown in FIG. 3B, strong specular reflection in some regions partially saturates the image and hinders observation of underlying tissue structures. In comparison, in the cross-polarized image, the specular reflection is removed allowing good visualization of the sub-epithelial structures as shown in FIG. 3C.

FIG. 4 shows images of the floor of mouth in a subject diagnosed with dysplasia in the region. The arrow in the fluorescence image, as shown in FIG. 4A, is pointed at an area with strong loss of fluorescence. The un-polarized (FIG. 4B) and polarized white light reflectance images (FIG. 4C) of the same area showed no obvious clinical abnormalities. Both of the white light images show the proposed resection tissue drawn by the surgeon following examination with an optical device of the present disclosure.

The potential of an optical device of the present disclosure to differentiate between normal and dysplastic oral tissue is demonstrated in FIGS. 3A and 4A. In contrast to the image from a normal subject, the autofluorescence signal from dysplastic tissue is overall much weaker due to the decrease in stromal collagen and elastin density in underlying epithelial tissue [11, 12]. Furthermore, under the routine white light illumination, no mucosal abnormalities are visible in the dysplastic tissue as shown in FIGS. 4B and 4C; yet under the fluorescence light illumination, a certain area with dysplasia appears very distinct from the sourrrounding mucosa as indicated with the arrow. The difference in contrast between the unpolarized and polarized white light images in FIGS. 3A and 3B also highlights the potential that the polarized light may be more useful for routine white light examination than unpolarized light.

Therefore, the present disclosure is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

1. Ferlay J, Bray F, Pisani P, Parkin D M. GLOBOCAN 2002: Cancer Incidence. Mortality and Prevalence Worldwide. IARC CancerBase No. 5, version 2.0., Lyon: IARC Press 2004.
2. American Cancer Society, “Cancer facts and figures 2007,” American Cancer Society, Atlanta, Ga. 2007.
3. Sankaranarayanan R, Black R J, Parkin D M. Cancer survival in developing countries. IARC Scientific Publication No. 145. Lyon: International Agency for Research on Cancer, 1998.
4. Wood N K, Goaz P W. Differential diagnosis of oral lesions. The C. V. Mosby Co: St. Louis; 1980. p. 105.
5. Lam S, MacAulay C, Hung J, LeRiche A, Profio E, and Palcic B, Detection of dysplasia and carcinoma in situ with a lung imaging fluorescence endoscope device, J. Thorac. Cardiovasc. Surg. 105, 1035-1040 1993.
6. Zeng H, McLean D, MacAulay C, and Lui H, Autofluorescence properties of skin and applications in dermatology, Proc. SPIE 4224, 366-373 2000.
7. Betz C S, Mehlmann M, Rick K, Stepp H, Grevers G, Baumgartner R, Leunig A. Autofluorescence imaging and spectroscopy of normal and malignant mucosa in patients with head and neck cancer. Lasers Surg Med. 1999; 25(4):323-34.7.
8. Paczona R, Temam S, Janot F, Marandas P, and Luboinski B, Autofluorescence videoendoscopy for photodiagnosis of head and neck squamous cell carcinoma, Eur. Arch. Otorhinolaryngol. 260, 544-548 (2003).
9. Onizawa K, Saginoya H, Furuya Y, Yoshida H, and Fukuda H, Usefulness of fluorescence photography for diagnosis of oral cancer, Int. J. Oral Maxillofac Surg. 28, 206-210 (1999).
10. Svistun E, Alizadeh-Naderi R, El-Naggar A, Jacob R, Gillenwater A, Richards-Kortum R, Vision enhancement system for detection of oral cavity neoplasia based on autofluorescence, Head & Neck, Volume 26, Issue 3, 2004.
11. Lane P, Gilhuly T, Whitehead P, Zeng H, Poh C, Ng S, Williams P, Zhang L, Rosin M, MacAulay C, Simple device for the direct visualization of oral-cavity tissue fluorescence, J of Biomed Optics, 11(2), 24006, 2006.
12. Pavlova I, Sokilov K, Drezek R, Malpica A, Follen M, and Richards-Kortum R, Microanatomical and biochemical origins of normal and precancerous cervical autofluorescence using laser-scanning fluorescence confocal microscopy, Photochem. Photobiol. 77, 550-555 (2003).

What is claimed is:

1. A device for examining tissue comprising:
a frame;
at least one light-emitting diode light source mounted on the frame for illuminating a tissue, wherein the at least one light-emitting diode light source is selected from the group consisting of a fluorescent light source, a polarized white light source, and an unpolarized white light source;
at least one loupe mounted on the frame for visually observing the tissue;
at least one filter disposed between the tissue and the loupe for filtering light reflected from the tissue; and
an energy source operably connected to the at least one light-emitting diode light source.

2. The device of claim 1 further comprising a camera mounted on the frame for capturing an image of the tissue and operably connected to the energy source.

3. The device of claim 1 wherein the frame is adapted to be worn on the head of a user.

4. The device of claim 1 wherein the frame is adapted to be held in the hand of a user.

5. The device of claim 1 wherein the camera is a charge-coupled device camera.

6. The device of claim 1 wherein the energy source is a battery.

7. The device of claim 1 wherein the energy source is a lithium-ion battery.

8. A system for visualizing tissue comprising:
a frame;
at least one light-emitting diode light source mounted on the frame for illuminating a tissue, wherein the at least one light-emitting diode light source is selected from the group consisting of a fluorescent light source, a polarized white light source, and an unpolarized white light source;
at least one loupe mounted on the frame for visually observing the tissue;
at least one filter disposed between the tissue and the loupe for filtering light reflected from the tissue;
a camera mounted on the frame for capturing an image of the tissue;
an energy source operably connected to the at least one light-emitting diode light source and the camera; and
a display monitor operably connected to the camera for receiving a signal produced by the camera.

9. The system of claim 8 further comprising a spectrometer operably connected to the camera for measuring the intensity of light reflected from the tissue.

10. A method for visualizing tissue comprising: illuminating a tissue with at least one light-emitting diode light source selected from the group consisting of a fluorescent light source, a polarized white light source, and an unpolarized white light source; and observing the tissue after illumination with the at least one light-emitting diode light source.

11. The method of claim 10 further comprising capturing an image of the illuminated tissue with a camera.

12. The method of claim 10 wherein the tissue is illuminated with a device according to claim 1.

13. The method of claim 11 wherein the camera is a charge-coupled device camera.

14. The method of claim 10 further comprising measuring the intensity of light reflected from the tissue.
